(19) United States
(12) Patent Application Publication
(10) Pub. No.: US 2022/0076821 A1
(43) Pub. Date: Mar. 10, 2022

(54) VITALS MONITORING PLATFORM FOR MULTIPLE USERS

(71) Applicant: Soma Health, Inc., Englewood, NJ (US)

(72) Inventors: Kyriakos Eric Mourkakos, Alpine, NJ (US); Dimitrios Sakalis, Flushing, NY (US); Sudarshan Walikar, Karnataka (IN); Manjunath Shivanna, Karnataka (IN); Sumukh Narendra, Karnataka (IN)

(21) Appl. No.: 17/169,277

(22) Filed: Feb. 5, 2021

(30) Foreign Application Priority Data
Sep. 5, 2020 (IN) 202031038386

(51) Int. Cl. G16H 40/67; A61B 5/0205; A61B 5/00; G16H 70/20

(52) U.S. Cl. CPC G16H 40/67; A61B 5/02055; A61B 5/7282; A61B 5/02405; A61B 5/0022; G16H 70/20; A61B 5/746

(57) ABSTRACT

A method including receiving, from a mobile device, a medical data indicative of one or more vital signs for a user of the mobile device, is provided. The vital signs include one of a body temperature, a pulse oximetry value, and a respiratory rate value, wherein the medical data includes a blood pressure value derived from one or more vital signs from the user. The method also includes finding a medical condition of the user based on the medical data; adding the medical data to a group data including vital signs for multiple users; and transmitting the medical condition to a second user, for treatment. A non-transitory, computer readable medium storing instructions and a system configured to perform the above method are also provided.

### CROSS-REFERENCE TO RELATED APPLICATIONS

The present disclosure is related and claims priority under 35 U.S.C. 119(b) to Indian Provisional Patent Application No. 202031038386, entitled VITALS MONITORING PLATFORM FOR MULTIPLE USERS, to Eric Mourkakos et-al. filed on Sep. 5, 2020, the contents of which are hereby incorporated by reference in their entirety, for all purposes.

### BACKGROUND

### Field

The present disclosure includes embodiments in the field of sharing and monitoring biometrics of multiple users and one or more groups of users. More specifically, the present disclosure includes embodiments in the field of monitoring vitals such as temperature, pulse oximetry, respiratory rate, heart rate, heart rate variation, resting heart rate, blood pressure, activity monitoring, and fall detection.

### Background Art

In order to measure temperature, pulse oximetry, respiratory rate, heart rate, heart rate variation, resting heart rate, blood pressure, activity (steps and calories), and fall detection, users are required to use multiple devices. Further, since each of these devices connects to its own mobile app, users have no choice but to install and use multiple applications to monitor the various biometrics. Additionally, the devices on the market today are all created for and targeted towards the individual user. That is, the data is not collected nor stored in a way that maximizes sharing with either a group or a physician.

### SUMMARY

In a first embodiment, a computer-implemented method includes receiving, from a mobile device, a medical data indicative of one or more vital signs for a user of the mobile device, the vital signs including one of a body temperature, a pulse oximetry value, and a respiratory rate value. The medical data includes a heart rate value, a heart rate variation value, a resting heart rate value, and a blood pressure value derived from one or more vital signs from the user. The computer-implemented method also includes finding a medical condition of the user based on the medical data, adding the medical data to a group data including vital signs for multiple users and transmitting the medical condition to a second user, for treatment.

In a second embodiment, a computer-implemented method includes receiving, from a sensor in close proximity of a user, a medical value indicative of a vital sign of the user. The computer-implemented method also includes deriving a user blood pressure based on the vital sign of the user, storing the medical value and a heart rate value, a heart rate variation value, a resting heart rate value, and a blood pressure value in a memory, detecting a mobile device in a vicinity of a radio-frequency antenna and transmitting the medical value to the mobile device using a radio-frequency signal from the radio-frequency antenna.

In another embodiment, a system, includes a memory, configured to store one or more instructions, and one or more processors configured to execute the instructions. The system is thus caused to receive, from a mobile device, a medical data indicative of one or more vital signs for a user of the mobile device. The vital sign includes one of a body temperature, a pulse oximetry value, and a respiratory rate value and the medical data includes a heart rate value, a heart rate variation value, a resting heart rate value, and a blood pressure value derived from one or more vital signs from the user. The system is also caused to add the medical data to a group data including vital signs for multiple users, to find a medical condition of the user based on the group data and to transmit the medical condition to a second user, for treatment.

In yet another embodiment, a non-transitory, computer readable medium stores instructions, which when executed by a processor in a computer cause the computer to perform a method. The method includes receiving, from a mobile device, a medical data indicative of one or more vital signs for a user of the mobile device, the vital signs including one of a body temperature, a pulse oximetry value, and a respiratory rate value. The medical data includes a heart rate value, a heart rate variation value, a resting heart rate value, and a blood pressure value derived from one or more vital signs from the user. The method also includes finding a medical condition of the user based on the medical data, adding the medical data to a group data including vital signs for multiple users and transmitting the medical condition to a second user, for treatment.

### BRIEF DESCRIPTION OF THE DRAWINGS

FIG. 1 illustrates an example architecture suitable for monitoring vital signs from multiple users and groups of users, according to some embodiments.

FIG. 2 is a block diagram illustrating an example server, client device, and wearable device from the architecture in FIG. 1, according to some embodiments.

FIG. 3 illustrates an example network including multiple users and groups of users of an application for monitoring vital signs hosted by a network server, according to some embodiments.

FIG. 4 illustrates a wearable device, a mobile device, a desktop device, and a server, configured for monitoring vital signs from multiple users and groups of users, according to some embodiments.

FIG. 5 illustrates aspects of a mobile application hosted by a network server in a mobile device, for monitoring vital signs from multiple users and groups of users, according to some embodiments.

FIGS. 6A, 6B, and 6C illustrate aspects of a desktop application hosted by a network server in a desktop device, for monitoring vital signs from multiple users and groups of users, according to some embodiments.

FIG. 7 is a flow chart illustrating steps in a method for collecting and processing vital signs information from multiple users and groups of users, according to some embodiments.

FIG. 8 is a flow chart illustrating steps in a method for collecting and processing vital signs information from a user, according to some embodiments.

FIG. 9 is a block diagram illustrating an example computer system with which the client and server of FIGS. 1 and 2 and the methods of FIGS. 7 and 8 can be implemented.

In the figures, elements and steps denoted by the same or similar reference numerals are associated with the same or similar elements and steps, unless indicated otherwise.

### DETAILED DESCRIPTION OF THE DRAWINGS

In the following detailed description, numerous specific details are set forth to provide a full understanding of the present disclosure. It will be apparent, however, to one ordinarily skilled in the art, that the embodiments of the present disclosure may be practiced without some of these specific details. In other instances, well-known structures and techniques have not been shown in detail so as not to obscure the disclosure.

### General Overview

In some embodiments, a computer-implemented method includes receiving, from a mobile device, a medical data indicative of one or more vital signs for a user of the mobile device. The vital signs include one of a body temperature, a pulse oximetry value, and a respiratory rate value, and the medical data includes a heart rate value, a heart rate variation value, a resting heart rate value, and a blood pressure value derived from one or more vital signs from the user. The computer-implemented method also includes adding the medical data to a group data including vital signs for multiple users, finding a medical condition of the user based on the group data, and transmitting the medical condition to a second user, for treatment.

Some embodiments include a wearable device that continuously monitors temperature, respiratory rate, pulse oximetry, heart rate, activity, and fall detection. In some embodiments, the wearable device links to the user's smartphone within seconds and continuously uploads data to the cloud via Bluetooth connectivity. The system may also be configured to alert users if vitals fall out of a given range.

In some embodiments, the wearable device has a companion mobile application and a web application that promotes group monitoring via a feature called Teams. The Teams feature enables users to continuously share their data in real time through a social network including one or more groups (or “teams”) including healthcare professionals. The social networking of multiple wearable devices, mobile devices, servers and other computers enables a powerful tool to monitor vitals and receive alerts should any metric fall out of a pre-determined range. In some embodiments, groups or teams in a social network as disclosed herein may include schools, nursing homes, businesses, and more, to monitor all of its members and ensure vitals are within a safe and normal range.

In some embodiments, the system includes a wearable device having high quality sensors to capture vital signs for individual users in real-time. These sensors may be tested and approved by various certifying bodies. Electronic components used in the wearable device are tested against various parameters such as fire, water, drop test, and battery charge compliance. The wearable device has an internal flash memory that stores vital measurements up to one month, and every parameter that is captured in the device is encrypted and stored in the built-in flash memory. The wearable device uses Bluetooth or any other wireless technology to transfer data from the wearable device to a mobile application installed in the mobile device for the user. The data transmission protocols used for sending data from device to app and app to cloud is HTTPS, which in turn encrypts data to the highest cryptographic standards.

### Example System Architecture

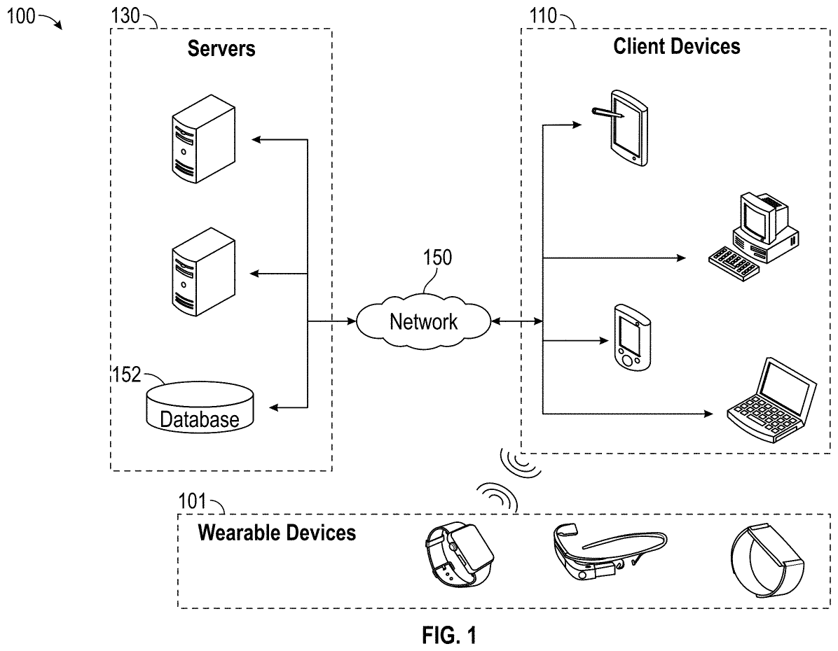

FIG. 1 illustrates an example architecture 100 suitable for monitoring vital signs from multiple users and groups of users, according to some embodiments. Architecture 100 includes servers 130 and client devices 110, and a database 152 communicatively coupled over a network 150. Architecture 100 also includes wearable devices 101 coupled with one or more client devices 110 via a close proximity wireless connection. One of the many servers 130 is configured to host a memory, including instructions which, when executed by a processor, cause the server 130 to perform at least some of the steps in methods as disclosed herein. In some embodiments, architecture 100 is configured to collect vital sign data from multiple users of wearable devices 101. The vital signs data is provided from a user by a wearable device 101 to a client device 110 via the wireless connection. This is done across a network of users, each having at least one wearable device 101 communicating with one client device 110. The vital signs data from the multiple users is provided to one or more of servers 130 for monitoring, processing, and storing in database 152.

Servers 130 may include any device having an appropriate processor, memory, and communications capability for hosting database 152, and a vitals monitoring engine. The vitals monitoring engine may be accessible by various client devices 110 over the network 150. Client devices 110 may include, for example, desktop computers, mobile computers, tablet computers (e.g., including e-book readers), mobile devices (e.g., a smartphone or PDA), or any other devices having appropriate processor, memory, and communications capabilities for accessing the vitals monitoring engine and the history log on one or more of servers 130. Network 150 can include, for example, any one or more of a local area network (LAN), a wide area network (WAN), the Internet, and the like. Further, network 150 can include, but is not limited to, any one or more of the following network topologies, including a bus network, a star network, a ring network, a mesh network, a star-bus network, tree or hierarchical network, and the like.

The wireless connection between wearable devices 101 and client devices 110 may include Bluetooth, low power Bluetooth (BLE), Wi-Fi, non contact near-field-communication (NFC), and the like. Accordingly, wearable devices 101 and client devices 110 may be equipped with software and hardware (e.g., antennas, radios, digital-to-analog converters—DAC—and analog-to-digital converters, and the like) to handle radio-frequency—RF—signals and signal processing. In addition to RF signal processing hardware and software, each of wearable devices 101 may include a host of sensors configured to measure vital signs of the user, such as temperature, pulse oximetry, respiration rate, activity monitors, fall detectors, heart rate, and blood pressure monitors. In some embodiments, the sensors in wearable devices 101 operate in close proximity to the user's body, or at least a part of the user's body, such as the skin, the eye, the skull, or any one of the user's limbs. In some embodiments, the wearable device may include an implanted device (e.g., intradermal), ingested device, or even a surgically implanted device (heart pacemakers and the like).

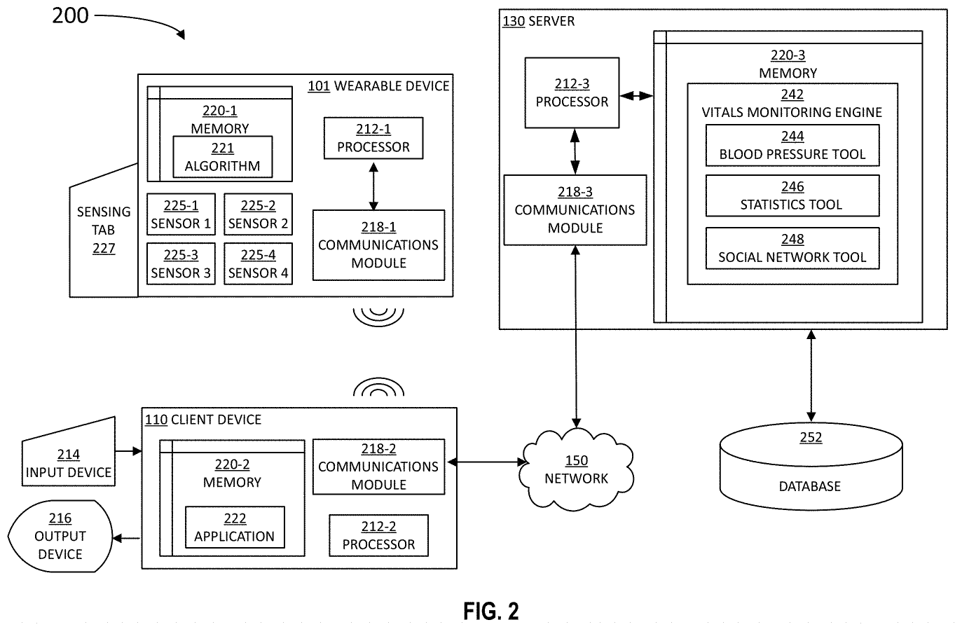

FIG. 2 is a block diagram 200 illustrating an example server 130, client device 110, and wearable device 101 from architecture 100, according to some embodiments. Wearable device 101, client device 110, and server 130 are communicatively coupled with each other and over network 150 via respective communications modules 218-1, 218-2, and 218-3 (hereinafter, collectively referred to as “communications modules 218”). Communications modules 218 are configured to interface with network 150 to send and receive information, such as data, requests, responses, and commands to other devices on the network. Communications modules 218 can be, for example, modems or Ethernet cards. In some embodiments, communications devices 218 may include software and hardware (e.g., antennas, radios, digital-to-analog converters—DAC—and analog-to-digital converters, and the like) to handle radio-frequency—RF—signals and signal processing. For example, in some embodiments, communication devices 218-1 and 218-2 may be configured for short-range or proximal wireless communication between wearable device 101 and client device 110 using RF signals. Wearable device 101 includes a processor 212-1 configured to execute instructions stored in a memory 220-1. Processor 212-1 is configured to execute instructions, such as instructions physically coded into processor 212-1, instructions received from software in memory 220-1, or a combination of both. In some embodiments, memory 220-1 is a flash memory that stores vital measurements up to one month, and every parameter that is captured in the device is encrypted and stored in the built-in flash memory. Client device 110 includes a processor 212-2 configured to execute instructions stored in a memory 220-2, and server 130 includes a processor 212-3 configured to execute instructions stored in a memory 220-3. Hereinafter, processors 212-1, 212-2, and 212-3 will be collectively referred to as “processors 212.” Likewise, memories 220-1, 220-2, and 220-3 will be collectively referred to as “memories 220.”

Wearable device 101 may also include multiple sensors 225-1, 225-2, 225-3, and 225-4 (hereinafter, collectively referred to as “sensors 225”). While block diagram 200 illustrates four sensors, the number of sensors included in wearable device 101 is not limiting, and some embodiments may include less than four sensors, while some embodiments may include more than four sensors. Sensors 225 may include a pressure sensor, a temperature sensor, a pulse oxygenation sensor, an accelerometer, a glucose sensor, and the like. Memory 220-1 may also include an algorithm 221 to process at least partially some of the data provided by sensors 225. For example, algorithm 221 may include a cuff-less blood pressure algorithm configured to determine a blood pressure value based on other data such as heart rate, temperature, and/or pulse oximetry data. In some embodiments, algorithm 221 may be configured to derive respiratory data from oximetry data and heart rate data. In some embodiments, wearable device 101 may include a sensing tab 227 configured to receive input from the user, such as contact with the skin, or any other body part of the user. Sensing tab 227 may be coupled to one or more of sensors 225, which provide a signal in response to contact of the user with sensing tab 227. In some embodiments, at least one of sensors 225 may have a separate sensing tab 227.

Sensors 225 may include hardware such as electrical, optical, and electro-optical components. Accordingly, sensors 225 may include RF generators, amplifiers, detectors, antennas, DAC circuitry, and the like. Further, sensors 225 may also include light emitting diodes (LED), lasers, and photodetectors, including lenses, waveguides, optical fibers, and the like. For example, a pulse oximetry sensor may include a laser or LED configured to emit light at one or two wavelengths, and a detector configured to detect light at the emitted wavelengths, collected after scattering off a portion of the user's body including blood vessels (e.g., in a pulse oximetry sensor, or in a glucose sensor).

Client device 110 may be coupled with an input device 214 and with an output device 216. Input device 214 may include a keyboard, a mouse, a pointer, or even a touch-screen display that a user may utilize to interact with client device 110. Likewise, output device 216 may include a display and a speaker with which the user may retrieve results from client device 130. Memory 220-2 may further include an application 222 including specific instructions which, when executed by processor 212-2, cause output device 216 to display features and portals hosted by server 130 including vital signs data from multiple users and multiple groups in network 150, as disclosed herein. Application 222 may be installed by server 130 and perform scripts and other routines provided by server 130.

Server 130 includes memory 220-3, processor 212-3, and communications module 218-3. Memory 220-3 includes a vitals monitoring engine 242 for sharing and monitoring biometrics of multiple users and one or more groups of users. Vitals monitoring engine 242 includes a blood pressure tool 244, a statistics tool 246, and a social network tool 248.

Blood pressure tool 244 may include some of the instructions used in algorithm 221 for processing sensor data and obtaining a blood pressure value or a respiratory measurement value. Statistics tool 246 is configured to process vital signs from one or multiple users and provide statistical values such as averages, standard deviations, expected values, and the like. Statistics tool 246 may also evaluate the vital signs of one user in the context of a group of users, and ascertain whether one or more of the vital signs of the user is unusually out of the norm or expectation. For example, when one of the vital signs of a user is out of the expected value beyond a pre-selected threshold value, statistics tool 246 may trigger vitals monitoring engine 242 to issue a health alert for the user. Social network tool 248 is configured to handle a group of users, attributes, and authorization credentials and privileges to access vital signs in database 252. Accordingly, social network tool 248 may also provide messaging or e-mail services between one or more users belonging to one or more groups. For example, one of the groups may include a health activity community (gymnastics or exercise group, and the like), and another group may include healthcare professionals. Accordingly, social network tool 248 may suggest and enable communication between members of the health activity community and the healthcare professionals group based on the vital signs data, statistics, and other group or individual attributes (e.g., habits, preferences, likes, dislikes, and even consumer history).

Database 252 stores the vital signs data provided from client device 110 to server 130. Database 252 may also include networking information about the user of client device 110 and wearable device 101, such as a network identification value and other attributes associated with the user (e.g., name, title, group, or “team” to which the user belongs, and role). Database 252 may also include, for each user, statistical information of vital signs. Database 252 may include the social network structure supported by social network tool 248, including attributes, roles, message logs—inbox, outbox—, and even consumer logs indicative of health habits of one or more users in one or more groups.

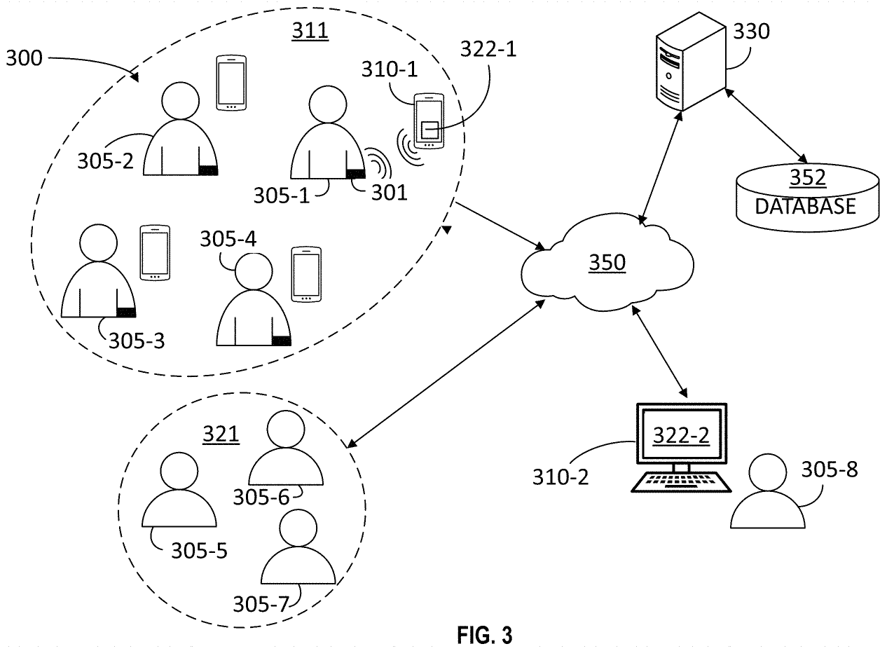

FIG. 3 illustrates an example social network 300 including multiple users 305-1, 305-2, 305-3, 305-4, 305-5, 305-6, 305-7, and 305-8, collectively referred to, hereinafter, as “users 305” and groups 311 and 321 of users of an application 322-2 for monitoring vital signs hosted by a network server 330, according to some embodiments. At least some of the users may have wearable devices, such as wearable device 301 for user 305-1, in group 311, also having a mobile device 310-1. Mobile device 310-1 includes a mobile application 322-1 to collect vital signs information from user 305-1, which is gathered by wearable device 301. In that regard, group 311 may include a health activity group, such as a gymnastics practicing team, and the like. Users 305-5, 305-6, and 305-7 may be associated with a group 321 that may be identified as a healthcare professional group. User 305-8 may access social network 300 in a database 352 by running application 322-2 in a desktop 310-2, hosted by server 330. Social network 300 is based on mobile devices 310-1, desktop 310-2, network server 330 and database 352 communicatively coupled through network 350 (cf. client devices 110, servers 130, database 152, and network 150).

Any one of users 305 may be part of one or more groups in social network 300. For example, any one of users 305-1 through 305-4 may be, in addition to a member of group 311 for a health activity group, a healthcare professional who is also a member of group 321. Likewise, user 305-8 accessing application 322 with desktop 310-2 may be a member of group 311, of group 321, or of neither.

In some embodiments, user 305-8 monitors the health records and vital signs for users 305 in group 311 and provides alerts and notifications to users 305 in group 321 (e.g., someone in group 311 may need medical intervention or advice from someone in group 321).

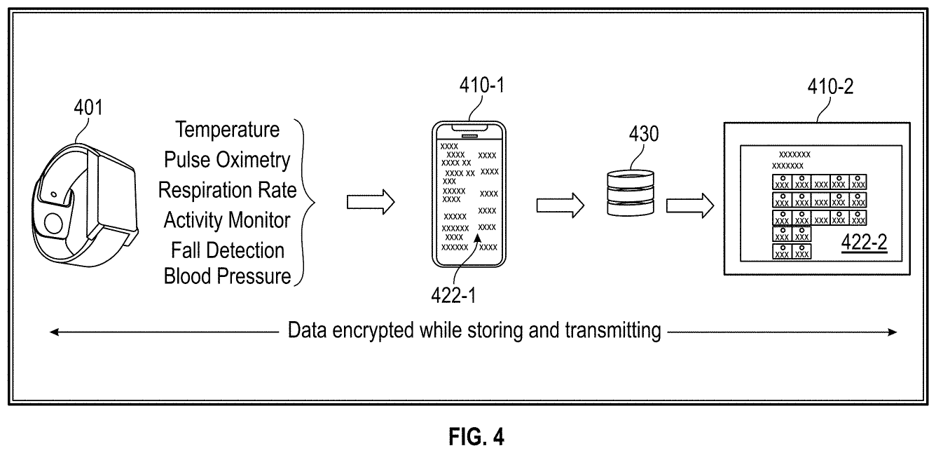

FIG. 4 illustrates a wearable device 401, a mobile device 410-1, a desktop device 410-2, and a server 430, configured for monitoring vital signs from multiple users and groups of users, according to some embodiments. Mobile device 410-1 may run an application 422-1 to collect vital signs data from wearable device 401. Application 422-1 also provides the data to server 430. An external user may access server 430 via application 422-2 in desktop device 410-2. In some embodiments, applications 422-1 and 422-2 (hereinafter, collectively referred to as “applications 422”) may ensure that the data is encrypted at all levels of communication, from wearable device 401, mobile device 410-1, and desktop device 410-2.

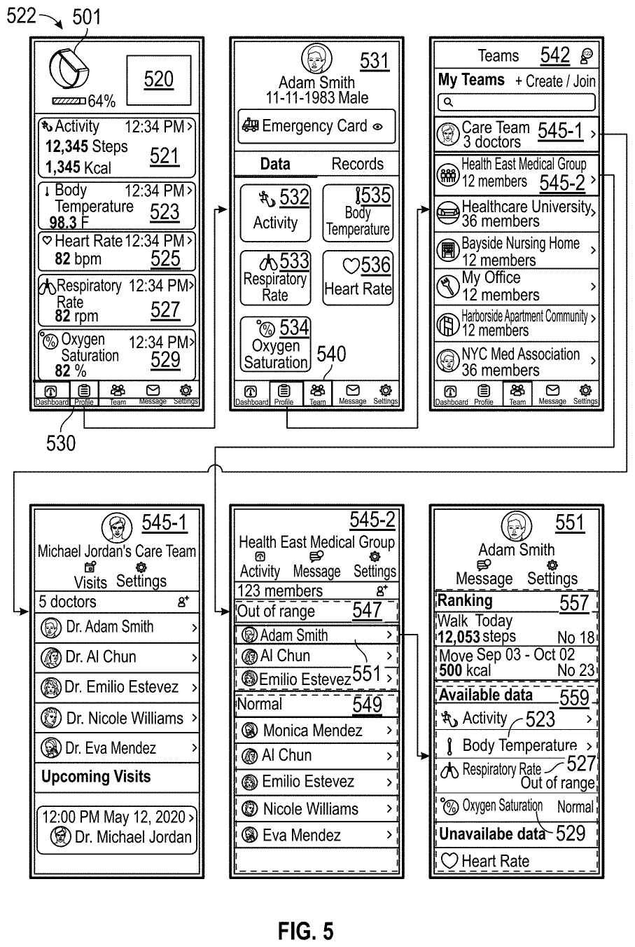

FIG. 5 illustrates aspects of a mobile application 522 hosted by a network server in a mobile device (e.g., servers 130, 330, and 430, client devices 110 and mobile devices 310 and 410, and applications 222, 322-1, and 422-1), for monitoring vital signs from multiple users and groups of users, according to some embodiments. Application 522 may be hosted by a remote server having access to a database as disclosed herein (e.g., servers 130, 330, and 430, and databases 152 and 252).

In one aspect, application 522 may have a pop-up display 520 indicating a power status of a wearable device 501 configured to provide vital signs of the user. Display 520 also includes a field 521 indicating features such as number of steps or calories consumed by the user, a body temperature 523, a heart rate 525, a respiratory rate 527, and an oxygen saturation value 529. Wearable device 501 may determine number of steps by performing analysis on a trace provided by an accelerometer, and the calories consumed by the user may also include an analysis of the accelerometer data combined with a known mass for the user. Heart rate (beats per minute—bpm—) 525 may also be provided by analysis of the photoplethysmogram (PPG) value (e.g., looking for a signal having a periodicity of about one minute). A PPG is an optically obtained plethysmogram that can be used to detect blood volume changes in the microvascular bed of tissue. In some embodiments, a PPG may be obtained by using a pulse oximeter which illuminates the skin and measures changes in light absorption at selected wavelengths. A respiratory rate (respirations per minute—rpm—) 527 may be determined by combining heart rate 525 with other values such as oxygen saturation 529, as well as analysis of the PPG signal.

Pop-up display 520 may include a bottom bar to access other aspects of application 522, such as a user profile 530, or a group management feature 540. The user profile 530 opens a display 531 illustrating multiple aspects about the user such as: activity 532, body temperature 535, respiratory rate 533, heart rate 536, and oxygen saturation 534. While pop-up display 520 illustrates instant vales of the above parameters (e.g., body temperature 523, heart rate 525, respiratory rate 527, and oxygen saturation 529), features 532, 533, 534, 535, and 536 are links to historical data and statistical data for those values (e.g., retrieved from the database).

The group management feature 540 opens a display 542 that list teams 545-1 and 545-2 (hereinafter, collectively referred to as “teams 545”). In some embodiments, the user is part of at least one of teams 545. In some embodiments, the user may select to follow at least one of teams 545. In some embodiments, the user may be authorized to follow at least one of teams 545 (e.g., the user is a physician for one or more members of the team, or is associated with a healthcare provider thereof). When the user accesses each one of teams 545, a list of the individual members is displayed, and the user may select any one of the members to view any of the related features (e.g., personal messages exchanged with that particular member, scheduled meetings or events involving that particular member, and other data about the member that the user is authorized to view). For example, in some embodiments, a display of team 545-2 may include an “out of range” listing 547, including team members that have one or more vital signs that are out of range as indicated by a statistics tool in the server (e.g., statistics tool 246). The display of team 545-2 may also include a “normal” listing 549, including team members that have all vital signs within a normal range as indicated by the statistics tool.

When the user taps into any of the members in teams 545, it may access information such as ranking 557 (e.g., ranking in terms of number of steps walked, calories burned, and the like). Other available data 559 may include current values of the vital signs: body temperature 523, respiration rate 527, and oxygen saturation 529. Each of the vital signs for team member 551 may include not indicating a status for the value such as “normal” or “out of range.”

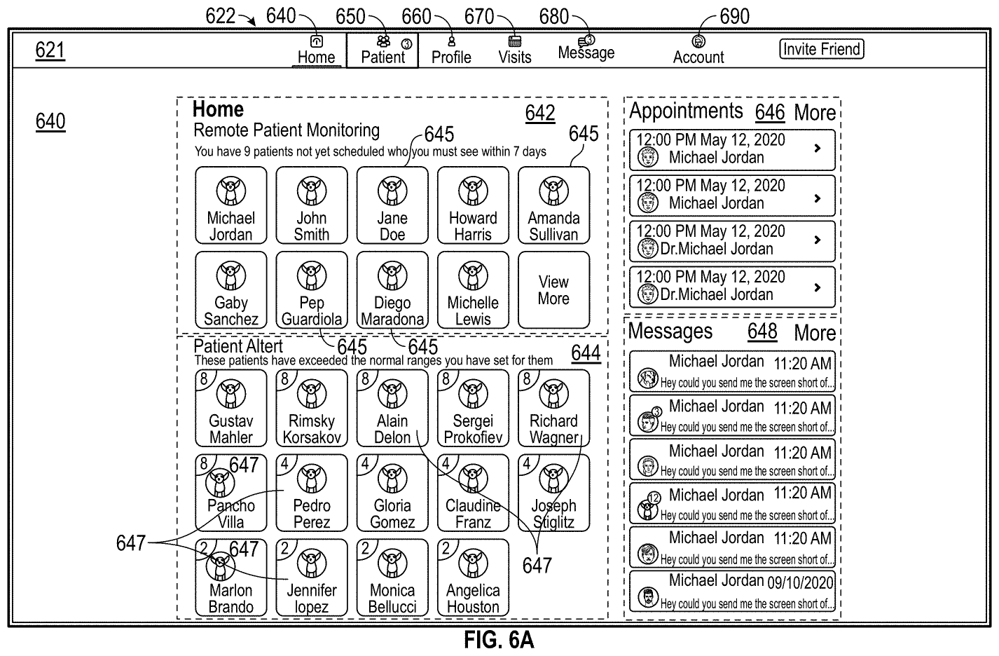

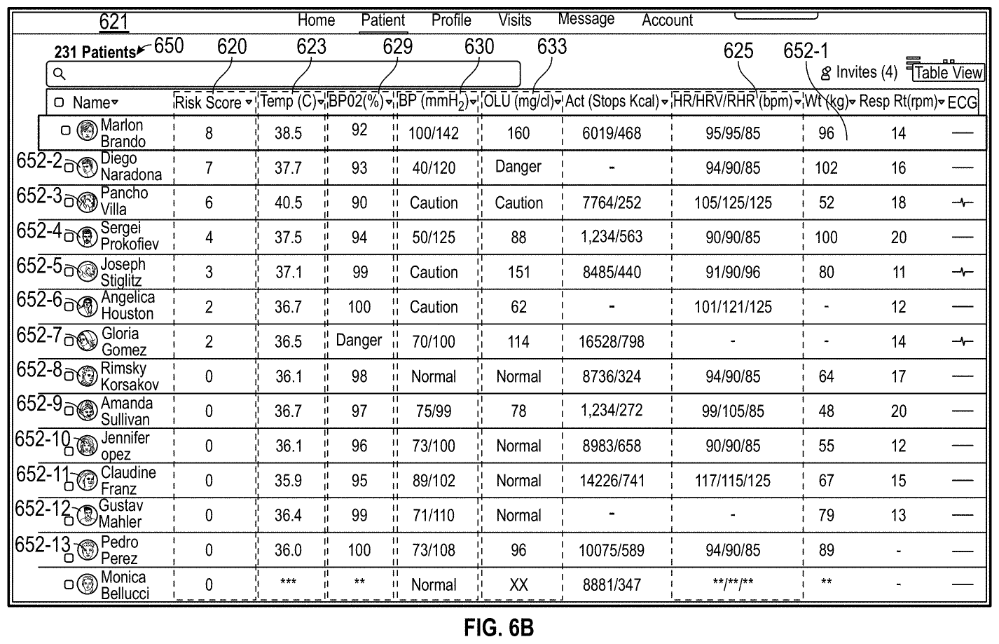

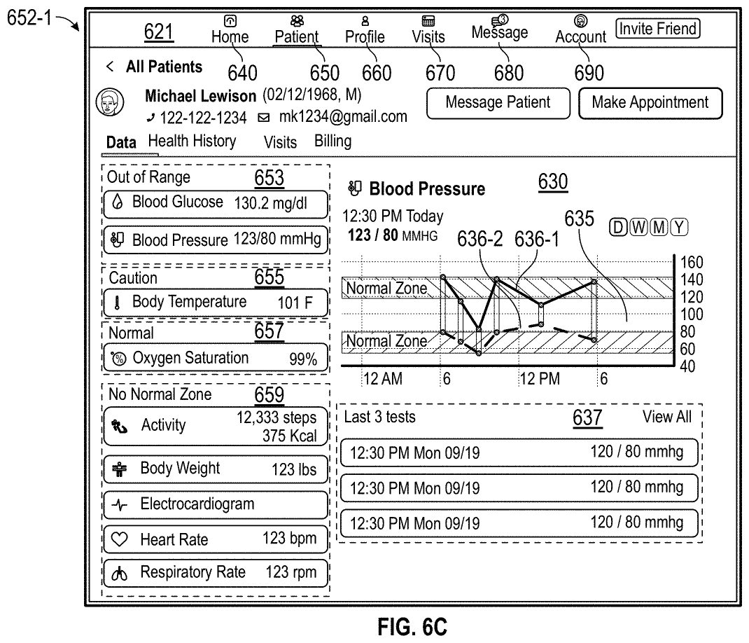

FIGS. 6A, 6B, and 6C illustrate aspects of a desktop application 622 hosted by a network server in a desktop device (e.g., servers 130, 330, and 430, client devices 110, 310-2, and 410-2), for monitoring vital signs from multiple users and groups of users, according to some embodiments. In some embodiments, a user accessing application 622 may be part of a social network supporting vital signs monitoring via a social networking tool in a vitals monitoring engine (e.g., user 305-8 in social network 300, vitals monitoring engine 242 and social network tool 248), with authorization and access privileges. A features bar 621 lists a home portal 640, a patient portal 650, a profile portal 660, a visits portal 670, a messages portal 680, and an account portal 690.

FIG. 6A illustrates home portal 640 including a remote patient monitoring field 642 and a patient alert field 644. Remote patient monitoring field 642 may list users 645 whose data may be accessed through home portal 640. Patient alert field 644 may list users 647 that have alerts because they have exceeded normal ranges in one or more vital signs that are set for them.

An appointments field 646 lists upcoming appointments for the user with other users in the network (e.g., when the user is a physician or healthcare professional and the other users are patients). A messages field 648 lists messages received and sent by the user to other users in the network.

FIG. 6B illustrates a patient portal 650, listing multiple patients 652-1, 652-2, 652-3, 652-4, 652-5, 652-6, 652-7, 652-8, 652-9, 652-10, 652-11, 652-12, and 652-13, hereinafter, collectively referred to as “patients 652”). Each of the patients have associated attributes, listed in columns, such as: a risk score 620, a body temperature 623, a cardiovascular sign 625, a pulse oximetry sign 629, a blood pressure sign 630, and a glucose level 633. In some embodiments, cardiovascular sign 625 may be provided as a list of three values: value 1/value 2/value 3. In some embodiments, value 1 indicates a heart rate (HR), value 2 indicates a heart rate variability (HRV), and value 3 indicates a resting heart rate (RHR).

FIG. 6C illustrates the display that a user (e.g., a physician) obtains when accessing data for another user (e.g., patient 652-1). The display may include a field 653 listing out of range vital signs for patient 652-1 (e.g., blood glucose and blood pressure). A field 655 may include cautionary vital signs (e.g., body temperature). A field 657 may include vital signs having normal values (e.g., oxygen saturation levels). A field 659 may include vital signs that, while not necessarily being out of range (cf. field 653), may be not normal, or at an undesirably high/low level (e.g., activity, body weight, electrocardiogram, heart rate, respiratory rate, and the like). In that regard, information on patient 652-1 and accessible to the physical through application 622 may include data collected at a clinic, using high-end, sophisticated laboratory equipment and procedures (electrocardiograms, magnetic resonance imaging—MRI—, and the like).

The patient display may also include a blood pressure field 630, including a chart 635 indicating a high value curve 636-1 (systolic blood pressure) and a low value curve 636-2 (diastolic blood pressure) over a period of time. Hereinafter, high value curve 636-1 and low value curve 636-2 will be referred to as “blood pressure curves 636.” In some embodiments, chart 635 may include a band illustrating a normal range value (e.g., for the systolic blood pressure and for the diastolic blood pressure). A field 637 may also include a listing of the last three tests of blood pressure for patient 652-1.

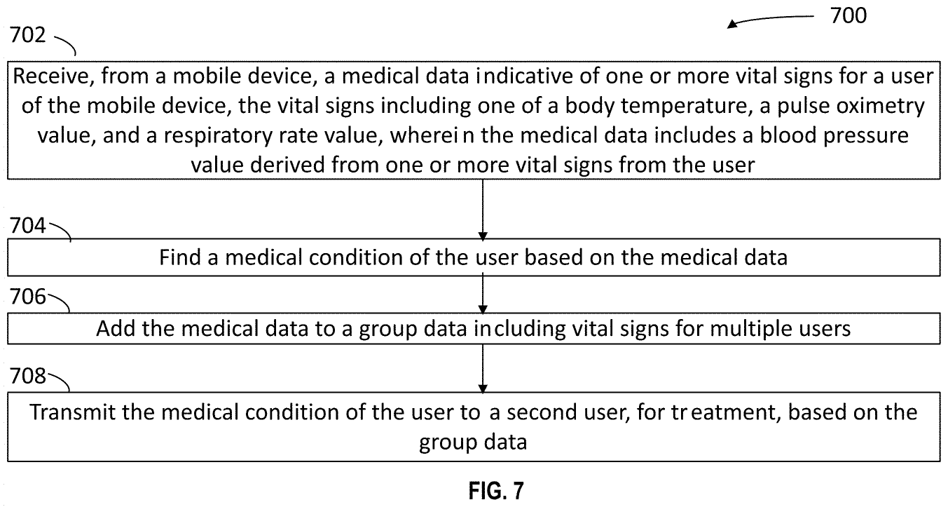

FIG. 7 is a flow chart illustrating steps in a method 700 for collecting and processing vital signs information from multiple users and groups of users, according to some embodiments. In some embodiments, at least one or more of the steps in method 700 may be partially or fully performed by a computer device including a processor, a memory, and a communications module (e.g., wearable device 101, client device 110, server 130, processors 212, memories 220, and communications modules 218). The communications modules may enable the computer devices to communicate with each other wirelessly or through a network (e.g., network 150). The processors may execute instructions stored as an application or an algorithm in the memories, to perform at least some of the steps in method 700 (e.g., algorithm 221 and application 222). More specifically, in some embodiments, at least some of the steps in method 700 may be performed by a processor executing instructions stored in a vitals monitoring engine having a blood pressure tool, a statistical tool, or a social network tool (e.g., vitals monitoring engine 242, blood pressure tool 244, a statistical tool 246, or social network tool 248), according to some embodiments. In some embodiments, the memories in method 700 may include a database accessible to the computer device via the network (e.g., databases 152 or 252). Furthermore, methods consistent with the present disclosure may include any of the steps in method 700 performed in any order. For example, in some embodiments, a method as disclosed herein may include at least one or more of the steps in method 700 performed in reverse order, simultaneously, almost simultaneously, or overlapping in time.

Step 702 includes receiving, from a mobile device, a medical data indicative of one or more vital signs for a user of the mobile device. The vital signs include one of: a body temperature, a pulse oximetry value, and a respiratory rate value. In some embodiments, the medical data also includes a blood pressure value derived from one or more vital signs from the user.

Step 704 includes finding a medical condition of the user based on the medical data. In some embodiments, the group data includes a statistical value associated with the vital signs, and step 704 includes evaluating that the medical data outlays the statistical value by a margin greater than a pre-selected threshold.

Step 706 includes adding the medical data to a group data including vital signs for multiple users.

Step 708 includes transmitting the medical condition of the user to a second user, for treatment, based on the group data. In some embodiments, the second user is a healthcare provider authorized to treat the user of the mobile device, and step 708 includes providing access to the second user to the vital signs. In some embodiments, the second user is a group database, and step 708 includes storing the medical condition and the vital signs in the group database. In some embodiments, the second user is a healthcare provider, and step 708 includes scheduling a healthcare inspection between the user of the mobile device and the healthcare provider. In some embodiments, step 708 includes transmitting an alert to the user of the mobile device when a metric for the medical condition falls out of a pre-determined range. In some embodiments, step 708 includes transmitting an alert to the second user when a metric for the medical condition falls out of a pre-determined range. In some embodiments, the group data is associated to at least one of a school, a nursing home, and a business, and the second user is an administrating server configured to ensure that the vital signs are within a pre-selected range. Accordingly, step 708 may include receiving, from the administrating server, a request to communicate with the user of the mobile device when one of the vital signs is outside of the pre-selected range. In some embodiments, step 708 includes receiving, from the mobile device, a data indicative of a battery charge compliance of a wearable device with the user of the mobile device. In some embodiments, step 708 includes verifying authorization credentials for the second user prior to transmitting the medical condition to the second user.

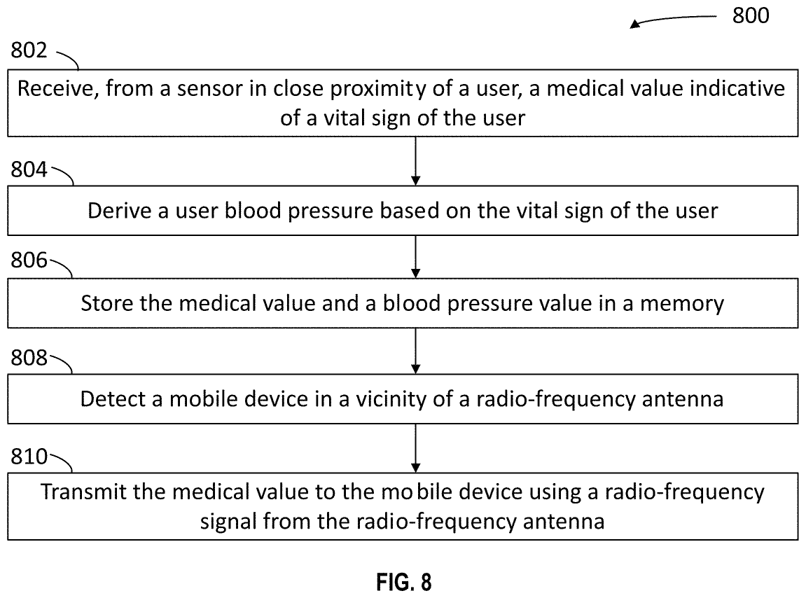

FIG. 8 is a flow chart illustrating steps in a method 800 for collecting and processing vital signs information from a user, according to some embodiments. In some embodiments, at least one or more of the steps in method 800 may be partially or fully performed by a computer device including a processor, a memory, and a communications module (e.g., wearable device 101, client device 110, server 130, processors 212, memories 220, and communications modules 218). The communications modules may enable the computer devices to communicate with each other wirelessly or through a network (e.g., network 150). The processors may execute instructions stored as an application or an algorithm in the memories, to perform at least some of the steps in method 800 (e.g., algorithm 221 and application 222). More specifically, in some embodiments, at least some of the steps in method 800 may be performed by a processor executing instructions stored in a vitals monitoring engine having a blood pressure tool, a statistical tool, or a social network tool (e.g., vitals monitoring engine 242, blood pressure tool 244, a statistical tool 246, or social network tool 248), according to some embodiments. In some embodiments, the memories in method 800 may include a database accessible to the computer device via the network (e.g., databases 152 or 252). Furthermore, methods consistent with the present disclosure may include any of the steps in method 800 performed in any order. For example, in some embodiments, a method as disclosed herein may include at least one or more of the steps in method 800 performed in reverse order, simultaneously, almost simultaneously, or overlapping in time.

Step 802 includes receiving, from a sensor in close proximity of a user, a medical value indicative of a vital sign of the user.

Step 804 includes deriving a user blood pressure based on the vital sign of the user. In some embodiments, the vital sign of the user includes a heart rate and a breathing rate, and step 804 includes determining the user blood pressure based on the heart rate and the breathing rate.

Step 806 includes storing the medical value and blood pressure value in a memory.

Step 808 includes detecting a mobile device in a vicinity of a radio-frequency antenna. In some embodiments, step 808 includes receiving a query from the mobile device to collect the medical value. In some embodiments, step 808 includes transmitting a beacon signal to the mobile device, and verifying that the mobile device is associated with the user.

Step 810 includes transmitting the medical value to the mobile device using a radio-frequency signal from the radio-frequency antenna. In some embodiments, step 810 includes deleting the medical value and the user blood pressure from memory after a pre-determined time lapse.

### Hardware Overview

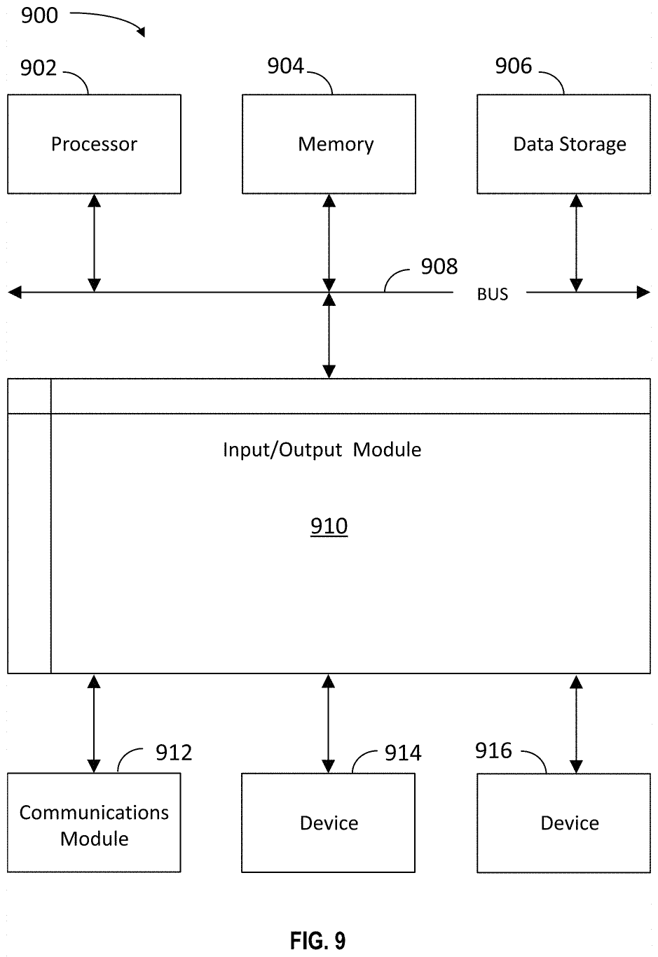

FIG. 9 is a block diagram illustrating an example computer system with which client devices 110, servers 130, wearable devices 101, and methods 700 and 800 can be implemented. In certain aspects, the computer system 900 may be implemented using hardware or a combination of software and hardware, either in a dedicated server, or integrated into another entity, or distributed across multiple entities.

Computer system 900 (e.g., client device 110 and server 130) includes a bus 908 or other communication mechanism for communicating information, and a processor 902 (e.g., processors 212) coupled with bus 908 for processing information. By way of example, the computer system 900 may be implemented with one or more processors 902. Processor 902 may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable entity that can perform calculations or other manipulations of information.

Computer system 900 can include, in addition to hardware, code that creates an execution environment for the computer program in question, e.g., code that constitutes processor firmware, a protocol stack, a database management system, an operating system, or a combination of one or more of them stored in an included memory 904 (e.g., memories 220), such as a Random Access Memory (RAM), a flash memory, a Read-Only Memory (ROM), a Programmable Read-Only Memory (PROM), an Erasable PROM (EPROM), registers, a hard disk, a removable disk, a CD-ROM, a DVD, or any other suitable storage device, coupled to bus 908 for storing information and instructions to be executed by processor 902. The processor 902 and the memory 904 can be supplemented by, or incorporated in, special purpose logic circuitry.

The instructions may be stored in the memory 904 and implemented in one or more computer program products, e.g., one or more modules of computer program instructions encoded on a computer-readable medium for execution by, or to control the operation of, the computer system 900, and according to any method well known to those of skill in the art, including, but not limited to, computer languages such as data-oriented languages (e.g., SQL, dBase), system languages (e.g., C, Objective-C, C++, Assembly), architectural languages (e.g., Java, .NET), and application languages (e.g., PHP, Ruby, Perl, Python). Instructions may also be implemented in computer languages such as array languages, aspect-oriented languages, assembly languages, authoring languages, command line interface languages, compiled languages, concurrent languages, curly-bracket languages, dataflow languages, data-structured languages, declarative languages, esoteric languages, extension languages, fourth-generation languages, functional languages, interactive mode languages, interpreted languages, iterative languages, list-based languages, little languages, logic-based languages, machine languages, macro languages, metaprogramming languages, multiparadigm languages, numerical analysis, non-English-based languages, object-oriented class-based languages, object-oriented prototype-based languages, off-side rule languages, procedural languages, reflective languages, rule-based languages, scripting languages, stack-based languages, synchronous languages, syntax handling languages, visual languages, wirth languages, and xml-based languages. Memory 904 may also be used for storing temporary variable or other intermediate information during execution of instructions to be executed by processor 902.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network. The processes and logic flows described in this specification can be performed by one or more programmable processors executing one or more computer programs to perform functions by operating on input data and generating output.

Computer system 900 further includes a data storage device 906 such as a magnetic disk or optical disk, coupled to bus 908 for storing information and instructions. Computer system 900 may be coupled via input/output module 910 to various devices. Input/output module 910 can be any input/output module. Exemplary input/output modules 910 include data ports such as USB ports. The input/output module 910 is configured to connect to a communications module 912. Exemplary communications modules 912 (e.g., communications modules 218) include networking interface cards, such as Ethernet cards and modems. In certain aspects, input/output module 910 is configured to connect to a plurality of devices, such as an input device 914 (e.g., input device 214) and/or an output device 916 (e.g., output device 216). Exemplary input devices 914 include a keyboard and a pointing device, e.g., a mouse or a trackball, by which a user can provide input to the computer system 900. Other kinds of input devices 914 can be used to provide for interaction with a user as well, such as a tactile input device, visual input device, audio input device, or brain-computer interface device. For example, feedback provided to the user can be any form of sensory feedback, e.g., visual feedback, auditory feedback, or tactile feedback; and input from the user can be received in any form, including acoustic, speech, tactile, or brain wave input. Exemplary output devices 916 include display devices, such as an LCD (liquid crystal display) monitor, for displaying information to the user.

According to one aspect of the present disclosure, the client device 110 and server 130 can be implemented using a computer system 900 in response to processor 902 executing one or more sequences of one or more instructions contained in memory 904. Such instructions may be read into memory 904 from another machine-readable medium, such as data storage device 906. Execution of the sequences of instructions contained in main memory 904 causes processor 902 to perform the process steps described herein. One or more processors in a multi-processing arrangement may also be employed to execute the sequences of instructions contained in memory 904. In alternative aspects, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system that includes a back-end component, e.g., a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. The communication network (e.g., network 150) can include, for example, any one or more of a LAN, a WAN, the Internet, and the like. Further, the communication network can include, but is not limited to, for example, any one or more of the following network topologies, including a bus network, a star network, a ring network, a mesh network, a star-bus network, tree or hierarchical network, or the like. The communications modules can be, for example, modems or Ethernet cards.

Computer system 900 can include clients and servers. A client and server are generally remote from each other and typically interact through a communication network. The relationship of client and server arises by virtue of computer programs running on the respective computers and having a client-server relationship to each other. Computer system 900 can be, for example, and without limitation, a desktop computer, laptop computer, or tablet computer. Computer system 900 can also be embedded in another device, for example, and without limitation, a mobile telephone, a PDA, a mobile audio player, a Global Positioning System (GPS) receiver, a video game console, and/or a television set top box.

The term “machine-readable storage medium” or “computer-readable medium” as used herein refers to any medium or media that participates in providing instructions to processor 902 for execution. Such a medium may take many forms, including, but not limited to, non-volatile media, volatile media, and transmission media. Non-volatile media include, for example, optical or magnetic disks, such as data storage device 906. Volatile media include dynamic memory, such as memory 904. Transmission media include coaxial cables, copper wire, and fiber optics, including the wires forming bus 908. Common forms of machine-readable media include, for example, floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH EPROM, any other memory chip or cartridge, or any other medium from which a computer can read. The machine-readable storage medium can be a machine-readable storage device, a machine-readable storage substrate, a memory device, a composition of matter affecting a machine-readable propagated signal, or a combination of one or more of them.

To illustrate the interchangeability of hardware and software, items such as the various illustrative blocks, modules, components, methods, operations, instructions, and algorithms have been described generally in terms of their functionality. Whether such functionality is implemented as hardware, software, or a combination of hardware and software depends upon the particular application and design constraints imposed on the overall system. Skilled artisans may implement the described functionality in varying ways for each particular application.

As used herein, the phrase “at least one of” preceding a series of items, with the terms “and” or “or” to separate any of the items, modifies the list as a whole, rather than each member of the list (e.g., each item). The phrase “at least one of” does not require selection of at least one item; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The word “exemplary” is used herein to mean “serving as an example, instance, or illustration.” Any embodiment described herein as “exemplary” is not necessarily to be construed as preferred or advantageous over other embodiments. Phrases such as an aspect, the aspect, another aspect, some aspects, one or more aspects, an implementation, the implementation, another implementation, some implementations, one or more implementations, an embodiment, the embodiment, another embodiment, some embodiments, one or more embodiments, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. Relational terms such as first and second and the like may be used to distinguish one entity or action from another without necessarily requiring or implying any actual such relationship or order between such entities or actions. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description. No claim element is to be construed under the provisions of 35 U.S.C. § 112, sixth paragraph, unless the element is expressly recited using the phrase “means for” or, in the case of a method claim, the element is recited using the phrase “step for.”

While this specification contains many specifics, these should not be construed as limitations on the scope of what may be described, but rather as descriptions of particular implementations of the subject matter. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially described as such, one or more features from a described combination can in some cases be excised from the combination, and the described combination may be directed to a subcombination or variation of a subcombination.

The subject matter of this specification has been described in terms of particular aspects, but other aspects can be implemented and are within the scope of the following claims. For example, while operations are depicted in the drawings in a particular order, this should not be understood as requiring that such operations be performed in the particular order shown or in sequential order, or that all illustrated operations be performed, to achieve desirable results. The actions recited in the claims can be performed in a different order and still achieve desirable results. As one example, the processes depicted in the accompanying figures do not necessarily require the particular order shown, or sequential order, to achieve desirable results. In certain circumstances, multitasking and parallel processing may be advantageous. Moreover, the separation of various system components in the aspects described above should not be understood as requiring such separation in all aspects, and it should be understood that the described program components and systems can generally be integrated together in a single software product or packaged into multiple software products.

The title, background, brief description of the drawings, abstract, and drawings are hereby incorporated into the disclosure and are provided as illustrative examples of the disclosure, not as restrictive descriptions. It is submitted with the understanding that they will not be used to limit the scope or meaning of the claims. In addition, in the detailed description, it can be seen that the description provides illustrative examples and the various features are grouped together in various implementations for the purpose of streamlining the disclosure. The method of disclosure is not to be interpreted as reflecting an intention that the described subject matter requires more features than are expressly recited in each claim. Rather, as the claims reflect, inventive subject matter lies in less than all features of a single disclosed configuration or operation. The claims are hereby incorporated into the detailed description, with each claim standing on its own as a separately described subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirements of the applicable patent law, nor should they be interpreted in such a way.

1. A computer-implemented method, comprising:
receiving, from a mobile device, a medical data indicative of one or more vital signs for a user of the mobile device, the vital signs comprising one of a body temperature, a pulse oximetry value, a heart rate value, a resting heart rate value, a heart rate variation value and a respiratory rate value, wherein the medical data comprises a blood pressure value derived from one or more vital signs from the user;
finding a medical condition of the user based on the medical data;
adding the medical data to a group data comprising vital signs for multiple users; and
transmitting the medical condition to a second user, for treatment.

2. The computer-implemented method of claim 1, wherein the group data comprises a statistical value associated with the vital signs, and wherein finding the medical condition of the user comprises evaluating that the medical data outlays the statistical value by a margin greater than a pre-selected threshold.

3. The computer-implemented method of claim 1, wherein the second user is a healthcare provider authorized to treat the user of the mobile device, and transmitting the medical condition comprises providing access to the second user to the vital signs.

4. The computer-implemented method of claim 1, wherein the second user is a group database, and transmitting the medical condition comprises storing the medical condition and the vital signs in the group database.

5. The computer-implemented method of claim 1, wherein the second user is a healthcare provider, further comprising scheduling a healthcare inspection between the user of the mobile device and the healthcare provider.

6. The computer-implemented method of claim 1, further comprising transmitting an alert to the user of the mobile device when a metric for the medical condition falls out of a pre-determined range.

7. The computer-implemented method of claim 1, further comprising transmitting an alert to the second user when a metric for the medical condition falls out of a pre-determined range.

8. The computer-implemented method of claim 1, wherein the group data is associated to at least one of a school, a nursing home, and a business, and the second user is an administrating server configured to ensure that the vital signs are within a pre-selected range, further comprising receiving, from the administrating server, a request to communicate with the user of the mobile device when one of the vital signs is outside of the pre-selected range.

9. The computer-implemented method of claim 1, further comprising receiving, from the mobile device, a data indicative of a battery charge compliance of a wearable device with the user of the mobile device.

10. The computer-implemented method of claim 1, further comprising verifying authorization credentials for the second user prior to transmitting the medical condition to the second user.

11. A computer-implemented method, comprising:
receiving, from a sensor in close proximity of a user, a medical value indicative of a vital sign of the user;
deriving a user blood pressure based on the vital sign of the user;
storing the medical value and a blood pressure value in a memory;
detecting a mobile device in a vicinity of a radio-frequency antenna; and
transmitting the medical value to the mobile device using a radio-frequency signal from the radio-frequency antenna.

12. The computer-implemented method of claim 11, wherein the vital sign of the user comprises, a heart rate value, a resting heart rate value, a heart rate variation value and a breathing rate, and deriving a user blood pressure comprises determining the user blood pressure based on the heart rate and the breathing rate.

13. The computer-implemented method of claim 11, wherein detecting the mobile device using a radio-frequency signal comprises receiving a query from the mobile device to collect the medical value.

14. The computer-implemented method of claim 11, wherein detecting a mobile device using a radio-frequency signal comprises transmitting a beacon signal to the mobile device, and verifying that the mobile device is associated with the user.

15. The computer-implemented method of claim 11, further comprising deleting the medical value and the user blood pressure from memory after a pre-determined time lapse.

16. A system, comprising:
a memory, configured to store one or more instructions; and
one or more processors configured to execute the instructions and cause the system to:
receive, from a mobile device, a medical data indicative of one or more vital signs for a user of the mobile device, the vital sign comprising one of a body temperature, a pulse oximetry value, a heart rate value, a resting heart rate value, a heart rate variation value, and a respiratory rate value, wherein the medical data comprises a blood pressure value derived from one or more vital signs from the user;
add the medical data to a group data comprising vital signs for multiple users;
find a medical condition of the user based on the group data; and
transmit the medical condition to a second user, for treatment.

17. The system of claim 16, further comprising a sensor remotely located in proximity with the user of the mobile device, wherein the sensor comprises a memory storing instructions, and a processor that executes the instructions to determine the blood pressure value from a heart rate ad a respiration rate of the user of the mobile device.

18. The system of claim 16, wherein the one or more processors further execute the instructions to request a personal identification information for the user from a third party identification server.

19. The system of claim 16, further comprising a sensor that is communicatively coupled with the mobile device via an encrypted communication protocol.

20. The system of claim 16, wherein the mobile device is in communication with the one or more processors via a wireless network.
